(19) United States
(12) Patent Application Publication
(10) Pub. No.: US 2024/0130985 A1
(43) Pub. Date: Apr. 25, 2024

(54) SOLID PHARMACEUTICAL TABLET

(71) Applicant: Genexa Inc., Atlanta, GA (US)

(72) Inventors: Max Spielberg, Miami, FL (US); David Johnson, Beverly Hills, CA (US)

(21) Appl. No.: 18/047,960

(22) Filed: Oct. 18, 2022

(51) Int. Cl. A61K 31/167; A61K 9/20

(52) U.S. Cl. CPC A61K 31/167; A61K 9/2013; A61K 9/2068

(57) ABSTRACT

A tablet includes an active material and a base material that carnauba wax and an oil.

### TECHNICAL FIELD

This application relates to formulations of tablets for the delivery of pharmaceuticals and to solid tablets for oral delivery to patients, people or animals. More particularly, it relates to formulations of solid tablets or caplets having improved characteristics and more natural ingredients.

### BACKGROUND

Oral drug administration typically has been the predominant route for drug delivery. A tablet is a pharmaceutical oral dosage form (oral solid dosage, or OSD) or solid unit dosage form. Tablets may be defined as the solid unit dosage form of medicament or medicaments with suitable excipients. Tablets comprise a mixture of active substances and excipients, usually in powder form, pressed or compacted from a powder into a solid dose. It is known to be the most popular route of drug administration due to the fact the gastrointestinal physiology offers more flexibility in dosage form design than most other routes. A major challenge for the pharmaceutical industry in drug development is to produce safe and efficient drugs with more natural ingredients.

Like clean food, clean medicines are thought to be minimally processed and free of things like synthetic, or artificial ingredients or preservatives. Although there are many tablet products available, there is a market segment that has been left unaddressed. Organic pharmaceutical tablets have been allusive in the marketplace.

Accordingly, there is always a need for an improved solid tablet. It is to this need, among others, that this application is directed.

### SUMMARY

This application provides a tablet having (a) an active material having an ingredient mixed therein, and (b) a base material of primarily carnauba wax.

In one aspect, the tablet can have an editable oil (e.g., a coating), dextrose, rice bran-based excipients such as rice bran and rice bran extract.

Another aspect includes a tablet having (a) an active material having a granulated ingredient mixed with a carnauba wax base material, and (b) suitable water-soluble sugars and/or sugar substitutes, and/or rice bran-based excipients like Nu-Rice® and Nu-Flow®. More particularly, one aspect includes a tablet having an edible oil, a dextrose, gum Arabic, rice bran-based excipients like rice bran and rice bran extract/hull.

Another aspect includes a tablet in which the hydrophobic material is selected from the group consisting of carnauba wax, beeswax, or a combination thereof.

Another aspect includes a tablet in which the active ingredient is a pharmaceutical ingredient, a nutritional supplement, or combinations thereof.

Another aspect includes a method of forming a tablet prepared by triturating active agents; combining the triturated component into a mixture with dextrose, rice brand; and compressing the combined mixture into a tablet. The tablet can then be coated with an edible oil.

Another aspect includes a tablet consisting of an active ingredient and a base material of carnauba wax, an edible oil coating (e.g., olive oil or sunflower oil), and rice bran or hull. The amount of carnauba wax is more than 50% or 60% or 70% or 80% or 90% of the base material.

Another aspect includes a tablet in which the active agent is greater than 90% of the tablet.

Another aspect includes a tablet in which the base material was primarily caramba wax with, had edible oil coating (e.g., palm or sunflower oil), had rice bran, had rice bran extract, had rice hull, and gum (arabic glycoproteins and polysaccharides, predominantly polymers of arabinose and galactose). The hardness was between 60N and 100N

### DETAILED DESCRIPTION

Persons with knowledge of solid dosage tablet formulations are familiar with the various excipients available to the formulator. These include those materials that are listed in the US Food and Drug Administration guide—Generally regarded as safe (GRAS).

One embodiment is a tablet having an active ingredient mixed with a base material or carnauba wax base material with an edible oil coating. The tablet can include suitable water-soluble sugars, rice bran-based excipients like Nu-Rice and Nu-Flow or rice hulls, and a hydrophobic material. This application discloses carnauba wax as a main non-active component of a tablet that can be effectively compressed into a tablet with an active ingredient.

The tablet includes a formulation of active ingredients mixed into carnauba wax base material. A tight particle size distribution may be suitable with the tablet. The process of tablet production can be either direct compaction or with the aid of extra steps (e.g., dry/wet granulation). The granulation process is utilized to adhere powder particles to form larger particles or granules, which can improve the quality of tableting depending on formulas. The average particle diameter (volume average particle diameter) of the particle group of a component can be between 30-1000 micrometers, or 70-500 micrometers, or 120-300 micrometers. For instance, the wet granulation process typically involves wet massing the solid ingredients of chewable formula with a liquid (e.g., water, ethanol, or isopropyl alcohol) to form wet aggregates. Then, the liquid is removed from the wet aggregates to form dry aggregates, followed by milling the dried aggregates to an appropriate size. Overwetting of granules in the wet granulation process can produce harder granules.

The solid tablet can have a tablet hardness of about 50 to 110N or about 60 to 90N. By setting the hardness of the initial product to the above lower limit value or more, it is possible to easily prevent tablet breakage or chipping in vibration or actual use during transfer, and by setting the hardness to the above preferable upper limit value or less, tablet disintegration is better.

In one embodiment, the tablet can be produced by a direct compaction process. In some embodiments, the method may include preparing the excipient base formula; blending the excipient base formula with at least one active ingredient at an appropriate weight ratio to obtain a solid formula. The formula can then be compressed into a tablet of appropriate size and shape. In some embodiments, all the desired ingredients may be mixed to assure homogeneity in an appropriate blender, followed by compressing the homogeneous mixture into a tablet of appropriate size and shape. Known-tableting process (e.g., mixing and blending) can be tailored according to the demand of tablet formulas.

Specific embodiments provide for the active drugs to be initially mixed with carnauba wax base material. When active agents are placed in the carnauba wax base material, the carnauba wax base material is heated to 180 degrees to assist with settling of the active agents. The active agents are then blended in the tablet.

In one embodiment, the tablet formulation is consisting of: an active ingredient, a carnauba wax base-material, organic rice bran (e.g., Nu Rice Bran®), wherein the carnauba wax is greater than 50% of the base material or greater than 60% of the base material or 70% of the base material, or 80% of the base material or 90% of the base material.

In another embodiment, the active ingredient in the tablet is an active pharmaceutical ingredient, a nutritional supplement, or combinations thereof.

In another embodiment, the active ingredient in the tablet is an active pharmaceutical ingredient.

In another embodiment, a method of forming a tablet as disclosed herein, said method comprising the steps of: preparing a trituration component of active ratio in a base material; combining said trituration component into a mixture with carnauba wax, organic dextrose, organic rice bran or rice bran extract; and compressing said combined mixture into a tablet.

In one embodiment, an edible oil can be a coating for the tablet. After tableting, the tablet may be subjected to a coating treatment as necessary for the purpose of improving the storage stability of the tablet. The coating treatment is not particularly limited, and a conventionally known method can be applied. For example, a method of applying a coating agent (an aqueous solution of a polymer or an edible oil) to the surface of a molded article after tableting and then may be dried.

An “active agent” is an agent (e.g., a compound) that is permitted or approved by the U.S. Food and Drug Administration, European Medicines Agency, or any successor entity thereof, for the oral treatment of a condition or disease. Suitable pharmaceutically active agents include, but are not limited to, analgesics, anti-inflammatory agents, antipyretics, antihistamines, antibiotics (e.g., antibacterial, antiviral, and antifungal agents), antidepressants, antidiabetic agents, antispasmodics, appetite suppressants, bronchodilators, cardiovascular treating agents (e.g., statins), central nervous system treating agents, cough suppressants, decongestants, diuretics, expectorants, gastrointestinal treating agents, anesthetics, mucolytics, muscle relaxants, osteoporosis treating agents, stimulants, nicotine, and sedatives.

In another embedment, the active agent includes herbal composition effective in the management of disorders. More particularly, the invention relates to an herbal composition effective in the management of disorders stress, pain, constipation and others. The invention further relates to the use of the herbal composition in preparation of food supplements, pharmaceuticals and nutraceuticals for management of disorders related to metabolic syndrome. Also provided is a method of treating disorders comprising administering to a subject in need thereof a therapeutically effective amount of the herbal composition of the present invention.

Examples of suitable analgesics, anti-inflammatories, and antipyretics include, but are not limited to, non-steroidal anti-inflammatory drugs (NSAIDs) such as propionic acid derivatives (e.g., ibuprofen, naproxen, ketoprofen, flurbiprofen, fenbufen, fenoprofen, indoprofen, ketoprofen, fluprofen, pirprofen, carprofen, oxaprozin, pranoprofen, and suprofen) and COX inhibitors such as celecoxib; acetaminophen; acetyl salicylic acid; acetic acid derivatives such as indomethacin, diclofenac, sulindac, and tolmetin; fenamic acid derivatives such as mefanamic acid, meclofenamic acid, and flufenamic acid; biphenylcarbodylic acid derivatives such as diflunisal and flufenisal; and oxicams such as piroxicam, sudoxicam, isoxicam, and meloxicam; isomers thereof; and pharmaceutically acceptable salts and prodrugs thereof.

Examples of antihistamines and decongestants, include, but are not limited to, bromopheniramine, chlorcyclizine, dexbrompheniramine, bromhexane, phenindamine, pheniramine, pyrilamine, thonzylamine, pripolidine, ephedrine, phenylephrine, pseudoephedrine, phenylpropanolamine, chlorpheniramine, dextromethorphan, diphenhydramine, doxylamine, astemizole, terfenadine, fexofenadine, naphazoline, oxymetazoline, montelukast, propylhexadrine, triprolidine, clemastine, acrivastine, promethazine, oxomemazine, mequitazine, buclizine, bromhexine, ketotifen, terfenadine, ebastine, oxatamide, xylomeazoline, loratadine, desloratadine, and cetirizine; isomers thereof; and pharmaceutically acceptable salts and esters thereof.

Examples of cough suppressants and expectorants include, but are not limited to, diphenhydramine, dextromethorphan, noscapine, clophedianol, menthol, benzonatate, ethylmorphone, codeine, acetylcysteine, carbocisteine, ambroxol, belladona alkaloids, sobrenol, guaiacol, and guaifenesin; isomers thereof; and pharmaceutically acceptable salts and prodrugs thereof. Examples of muscle relaxants include, but are not limited to, cyclobenzaprine and chlorzoxazone metaxalone, orphenadrine, and methocarbamol; isomers thereof; and pharmaceutically acceptable salts and prodrugs thereof. Examples of stimulants include, but are not limited to, caffeine. Examples of sedatives include, but are not limited to sleep aids such as antihistamines (e.g., diphenhydramine), eszopiclone, and zolpidem, and pharmaceutically acceptable salts and prodrugs thereof. Examples of appetite suppressants include, but are not limited to, phenylpropanolamine, phentermine, and diethylcathinone, and pharmaceutically acceptable salts and prodrugs thereof.

The percentages of active ingredients present will vary depending upon the desired characteristics of the particular product. Typically, the base material is present in a total concentration of at least about 0.001% by weight of the composition, such as in a range from about 0.001% to about 20%. In some embodiments, the base material and inactive ingredients is present in a concentration from about 0.1% w/w to about 10% by weight, such as, e.g., from about from about 0.5% w/w to about 10%, from about 1% to about 10%, from about 1% to about 5% by weight, based on the total weight of the composition. In some embodiments, the base material is present in a concentration of from about 0.001%, about 0.01%, about 0.1%, or about 1%, up to about 20% by weight, such as, e.g., from about from about 0.001%, about 0.002%, about 0.003%, about 0.004%, about 0.005%, about 0.006%, about 0.007%, about 0.008%, about 0.009%, about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5% about 0.6%, about 0.7%, about 0.8%, or about 0.9%, to about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20% by weight, based on the total weight of the composition. In other embodiments, the base material is greater than 0.1%, 1%, 2%, 4%, 10%, 20%, 30%, 40%, 50%, 60%, 70%, 80%, 90%, and 95% by weight, based on the total weight of the composition. In other embodiments, the amount of active agent can be up to about 90 to 99% of the weight of the composition.

Examples of anesthetics (e.g., for the treatment of sore throat) include, but are not limited to dyclonine, benzocaine, and pectin and pharmaceutically acceptable salts and prodrugs thereof.

In one embodiment, the pharmaceutically active agent included within the tablet is selected from phenylephrine, dextromethorphan, pseudoephedrine, acetaminophen, cetirizine, aspirin, nicotine, ranitidine, ibuprofen, ketoprofen, loperamide, famotidine, calcium carbonate, simethicone, chlorpheniramine, methocarbomal, chlophedianol, ascorbic acid, pectin, dyclonine, benzocaine and menthol, and pharmaceutically acceptable salts and prodrugs thereof.

In another embodiment, the tablet may further contain a lubricant.

The active agent may also be present in the form of pharmaceutically acceptable salts, such as acidic/anionic or basic/cationic salts. Pharmaceutically acceptable acidic/anionic salts include, and are not limited to acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate, pantothenate, phosphate/diphospate, polygalacturonate, salicylate, stearate, subacetate, succinate, sulfate, tannate, tartrate, teoclate, tosylate and triethiodide. Pharmaceutically acceptable basic/cationic salts include, and are not limited to aluminum, benzathine, calcium, chloroprocaine, choline, diethanolamine, ethylenediamine, lithium, magnesium, meglumine, potassium, procaine, sodium and zinc

The term chewing, in the context of this application, means to signify that the pulverizing or grinding is being performed by a patient's or subject's teeth, or gums. There is no intent to signify any degree of force required or generated by the teeth or gums. The requirement is that the force used to produce the pulverized granules, particles, powder and the like, is sufficient to disrupt the tablet.

The term “pharmaceutical compositions” comprise a carnauba wax, an active ingredient (preferably one, two or three) and at least one pharmaceutically acceptable excipient.

The term “granules” do also cover micro-granules. The granules can be used for direct administration or further processed into tablets or mini-tablets.

The term “tablet” covers any type of tablet resulting from the compression or compaction of powders, granules (obtained by wet or dry granulation, tableting, melt extrusion), mini-tablets, micro granules, pellets, but refers preferably a direct compressed tablet.

The term “compressed” covers any physical compaction process resulting in solid dosage units.

The term “organic,” in the context of this application, refers to the way agricultural products are grown and processed. While the regulations vary from country to country, in the U.S., organic crops must be grown without the use of synthetic herbicides, pesticides, and fertilizers, or bioengineered genes (GMOs). The tablet formation preferably includes ingredients that must be grown without the use of synthetic herbicides, pesticides, and fertilizers, or bioengineered genes (GMOs)

### EXAMPLES

### Example 1 (Acetaminophen)

A solid tablet was prepared having acetaminophen and a carnauba wax base material. The base material was primarily caramba wax with, had edible oil coating (e.g., palm or sunflower oil), had rice bran, rice bran extract, rice hull, and gum (arabic glycoproteins and polysaccharides, predominantly polymers of arabinose and galactose). The active material was acetaminophen. The base material and the active material are compressed into tablets and exhibited satisfactory friability. The active agent is between 50-90% by weight of the tablet. The base material contained over 60% caramba wax by weight of the base material. Rice bran/hull or rice bran extract, oils and gum were the remaining agents of the base material.

### Example 2 (Acetaminophen)

A tablet, a pharmaceutical formulation, or a pharmaceutical composition, which the base material comprises more than 65%, or more than 80%, or more than 90%, or more than 95%, or more than 98%, by weight on a dry weight basis of carnauba wax.

### Example 3 (Ibuprofen)

A tablet, a pharmaceutical formulation, or a pharmaceutical composition has the ingredients in Example 1 and the active ingredient is Ibuprofen.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

What is claimed:

1. A tablet formulation comprising: (a) an active material, and (b) a base material comprising the carnauba wax, wherein the carnauba wax is greater than 50% of the base material.

2. A tablet formulation of claim 1, wherein the carnauba wax is greater than 60% of the base material.

3. A tablet formulation of claim 1, wherein the carnauba wax is greater than 70% of the base material.

4. A tablet formulation of claim 1, wherein the carnauba wax is greater than 80% of the base material.

5. A tablet formulation of claim 1, further comprising an edible oil coating.

6. A tablet formulation of claim 1, wherein hardness of the tablet formulation is between 50N and 90N.

7. A tablet formulation of claim 1, wherein the base material comprises rice bran.

8. A tablet formulation of claim 1, wherein the active ingredient is a nutritional supplement.

9. A tablet formulation of claim 1, wherein the tablet is formed by direct compression.

10. A tablet formulation of claim 1, wherein the active agent is an active pharmaceutical ingredient.

11. A table formulation of claim 1, wherein the active agent is acetaminophen.

12. A method of forming a tablet, comprising the steps of:
a. preparing a base material of carnauba wax and an active agent, wherein the active agent is granulated;
b. combining the base material and the active agent into a mixture; and
c. compressing said combined mixture into a tablet.

13. A method of claim 12, wherein the mixture has a sugar, an edible oil, and a rice bran.

14. A method of claim 12, wherein the mixture is a flavoring agent.

15. A method of claim 12, wherein the carnauba wax is greater than 50% of the base material.

16. A base material for a tablet comprising: (a) carnauba wax and (b) an oil, wherein the carnauba wax is greater than 50% of the base material.

17. A base material for a tablet of claim 16, the amount of the carnauba wax is greater than 70% of the base material.

18. A base material for a tablet of claim 16, wherein said active ingredient in the tablet is an active pharmaceutical ingredient, a nutritional supplement, or combinations thereof.

19. A base material for a tablet of claim 16, wherein the amount of the carnauba wax is greater than 60% of the base material.

20. A base material for a tablet of claim 16, further comprising rice bran or rice bran extract, wherein the caramba wax is between 50 and 80% by weight of the base material, the rice bran or rice bran extract is between 1 and 25% by weight of the base material.

21. A tablet formulation comprising essentially: (a) an active material comprising an active agent and (b) a base material comprising carnauba wax and an oil, wherein the caramba wax is between 50 and 80% by weight of the base material, and the active agent is greater than 90% by weight of the tablet wherein hardness of the tablet formulation is between 50N and 90N.

22. A tablet formulation of claim 20, wherein the active agent is acetaminophen.
